(19) United States
(12) Plant Patent Application Publication
(10) Pub. No.: US 2003/0126664 P1
(43) Pub. Date: Jul. 3, 2003

(54) Petunia plant named 'Sunbelrikupi'

(76) Inventor: Yasuyuki Murakami, Gamo-gun (JP)

Correspondence Address: BURNS DOANE SWECKER & MATHIS L L P, POST OFFICE BOX 1404, ALEXANDRIA, VA, 22313-1404, US

(21) Appl. No.: 10/327,017

(22) Filed: Dec. 24, 2002

(30) Foreign Application Priority Data
Dec. 28, 2001 (JP) 14309

(51) Int. Cl. A01H 5/00

(52) U.S. Cl. PLT/356

(57) ABSTRACT

Disclosed herein is a new and distinct variety of Petunia plant having a decumbent growth habit. The Petunia plant has abundant branching particularly with respect to secondary branching, and great profusion of blooms with the entire plant remaining in bloom for a considerable period of time. The flowers are single and small with the petals having a vivid reddish purple color. The base color of the corolla throat is brilliant greenish yellow. The outside color of the corolla throat is light greenish yellow. The plant exhibits high resistance to rain, heat, drought and diseases such as powdery mildew.

### BACKGROUND OF THE VARIETY

The petunia is a very popular plant that is used for flower bedding and potting in the summer season. There are only a few small flower petunias, such as, ‘SUNTORY SP-R’ (U.S. Plant Pat. No. 9557), ‘SUNBERUBU’ (U.S. Plant Pat. No. 9754), ‘SUNBELCHIPI’ (U.S. Plant Pat. No. 10,355) and ‘SUNBELUKI’ (U.S. Plant Pat. No. 11,558). These petunias are of the semi-decumbent type and have a medium plant height, abundant branching, and a high resistance to heat, rain and disease. However there are only a few varieties having a decumbent habit, a great profusion of flowers, a vivid reddish purple flower color, a very small flower size and a high resistance to rain, heat, cold and disease. Accordingly, this invention is aimed at obtaining a new variety having vivid reddish purple petals, and very small flowers combined with the above features.

The new variety of Petunia plant according to this invention originated from crossing of ‘SUNBELCHIPI’ (female) variety (U.S. Plant Pat. No. 10355) and ‘SUNBELKUPI’ (male) variety (U.S. Plant Pat. No. 10287).

Initially, crossing was conducted with the ‘SUNBELCHIPI’ variety as female parent and the ‘SUNBELKUPI’ variety as pollen parent in May 1996 at the Oumi R&D Center of SUNTORY Ltd., located at 863-1, Aza-Iketani, Omori-cho, Youkaichi-shi, Shiga-ken, Japan. From that crossing 50 seedlings were obtained and five seedlings were selected in view of their semi-erect growth habit and vivid reddish purple colored petals, at the end of September 1997. These five seedlings were grown and were tested during potting and bedding trials. Only one plant of the present invention was selected in September 1998. The selected plant was propagated by cutting and then grown, in pot, in a trial in a greenhouse and in a field at the Omi R&D Center from April to November 1999. The botanical characteristics of the finally selected plant were examined using a similar variety, ‘SUNBELKUPI’ (U.S. Plant Pat. No. 10287), for comparison. As a result, it was concluded that this new Petunia is distinguishable from any other variety whose existence is known to us and is uniform and stable in its characteristics. This new variety of Petunia plant was named ‘SUNBELRIKUPI’.

In the following description, the color information is in accordance with The R.H.S. Color Chart of The Royal Horticultural Society, London, England. A color chart based on The Japan Color Standard for Horticultural Plants (J.H.S. Color Chart) is also added for reference.

The ‘SUNBELCHIPI’, used as female parent and a comparison in the obtaining of this new variety ‘SUNBELRIKUPI’, was previously filed in Japan (Seeds and Seedlings law Registration No. 7232) and U.S.A. (U.S. Plant Pat. No. 10355). The main botanical characteristics thereof are as follows:

Plant:

Growth habit.—Semi-decumbent.

Plant height.—15-16 cm.

Spreading area of plant.—The stem extends to a length of 11-13 cm from the base.

Blooming period.—April to late October in the southern Kanto area, Japan. The plant shape does not change throughout this period.

Stem:

Thickness.—2.3 mm.

Pubescence.—Present.

Branching.—Abundant with the superior branching propensity, especially secondary branches.

Length of internode.—1.4 cm.

Leaf:

Shape.—Lanceolate.

Length.—5.1 cm.

Width.—1.6 cm.

Color.—Dark yellow green (R.H.S. 146A, J.H.S. 3508).

Thickness.—0.2-0.4 mm.

Pubescence.—Sparse.

Leaf attaching angle to stem.—Slanted upward to horizontal.

Flower:

Facing direction.—Horizontal.

Type.—Single.

Shape.—Funnel-shape, with five-fissured limb.

Shape of petal tip.—Round.

Waving of petal.—Weak.

Lobation of petal.—Shallow.

Diameter.—2.8-2.9 cm.

Color.—Petal: Vivid purplish red (R.H.S. 57C, J.H.S. 9707). Bottom color of corolla throat: Strong yellow (R.H.S. 9A, J.H.S. 2513). Outside color of corolla tube: Light greenish yellow (R.H.S. 5C, J.H.S. 2904).

Reproductive organs.—1 normal pistil and 5 normal stamens.

Fertility.—Fertile, but self-incompatible.

Peduncle.—0.7-0.8 mm in thickness, and 1.9 cm in length.

Physiological and ecological characteristics.—High resistance to heat, rain, drought and diseases such as powdery mildew. The resistance to heat and rain is very strong.

Blooming: A bloom commonly lasts approximately 10 days on the plant. Pinching is not necessary to ensure continuous blooming; however, it does tend to enhance bloom production.

The ‘SUNBELKUPI’, used as male parent in obtaining this new variety ‘SUNBELRIKUPI’, was previously filed in Japan (Seeds and Seedlings law Registration No. 7230) and U.S.A. (U.S. Plant Pat. No. 10287). The main botanical characteristics thereof are as follows:

Plant:

Growth habit.—Decumbent.

Plant height.—5.8-6.1 cm.

Spreading area of plant.—The stem extends to a length of 18-19 cm from the base.

Blooming period.—April to late October in the southern Kanto area, Japan. The plant shape does not change throughout this period.

Stem:

Thickness.—1.2 mm.

Pubescence.—Sparse.

Branching.—Over abundant with a superior branching propensity, especially secondary branches.

Length of internode.—1.3 cm.

Leaf:

Shape.—Lanceolate.

Length.—3.5 cm.

Width.—1.0 cm.

Thickness.—0.2-0.3 mm.

Pubescence.—Sparse.

Color.—Dark green (R.H.S. 137B, J.H.S. 3707).

Leaf attaching angle to stem.—Slanted upward to horizontal.

Flower:

Facing direction.—Slanted upward to horizontal.

Type.—Single.

Shape.—Funnel-shape, with five-fissured limb.

Shape of petal tip.—Round.

Waving of petal.—Weak.

Lobation of petal.—Shallow.

Diameter.—2.7-2.9 cm.

Color.—Petal: Vivid reddish purple (R.H.S. 74A, J.H.S. 9207). Bottom color of corolla throat: Bright greenish yellow (R.H.S. 3A, J.H.S. 2905). Outside color of corolla tube: Pale yellow green (R.H.S. 1D, J.H.S. 3102).

Reproductive organs.—1 normal pistil and 5 normal stamens.

Fertility.—Fertile, but self-incompatible.

Peduncle.—0.7-0.8 mm in thickness, and 1.8 cm in length.

Physiological and ecological characteristics.—High resistance to heat, rain, drought and diseases such as powdery mildew. The resistance to heat and rain is very strong.

Blooming: A bloom commonly lasts approximately 10 days on the plant. Pinching is not necessary to ensure continuous blooming; however, it does tend to enhance bloom production.

### SUMMARY OF THE VARIETY

This new variety of Petunia plant ‘SUNBELRIKUPI’ has a decumbent habit with abundant branching, with single and small vivid reddish purple color flowers and forms a great profusion of blooms with the whole plant remaining in bloom for a considerable period of time. The plant has high tolerances to cold and heat, high resistance to pests and diseases, particularly powdery mildew, and high resistance to rain.

### BRIEF DESCRIPTION OF THE PHOTOGRAPHS

FIG. 1 is a photograph of flowers and leaves of the new variety of Petunia plant ‘SUNBELRIKUPI’.

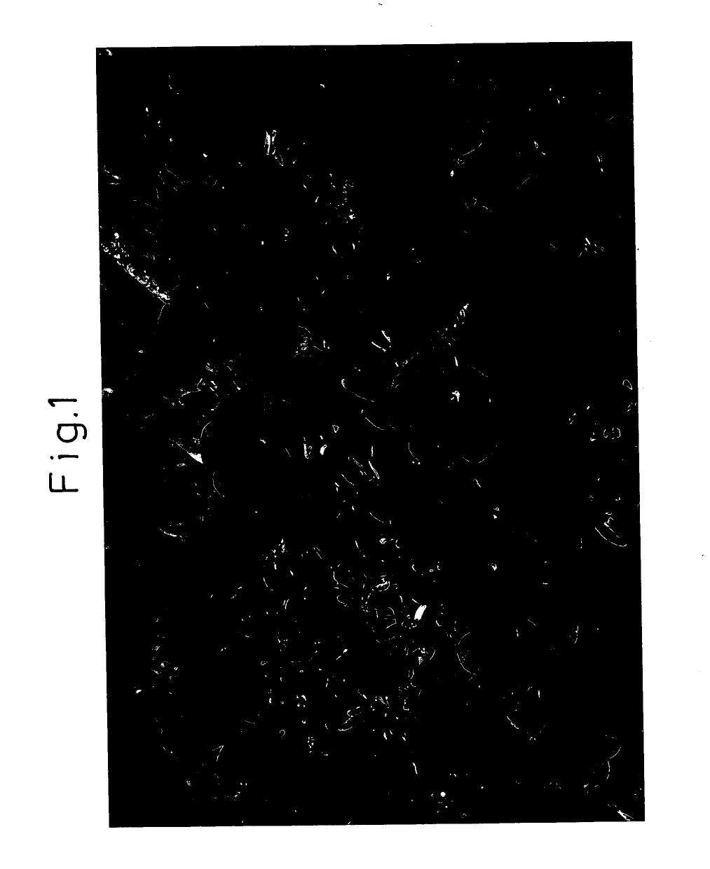

FIG. 2 is a photograph of the new variety of Petunia plant ‘SUNBELRIKUPI’ in a pot.

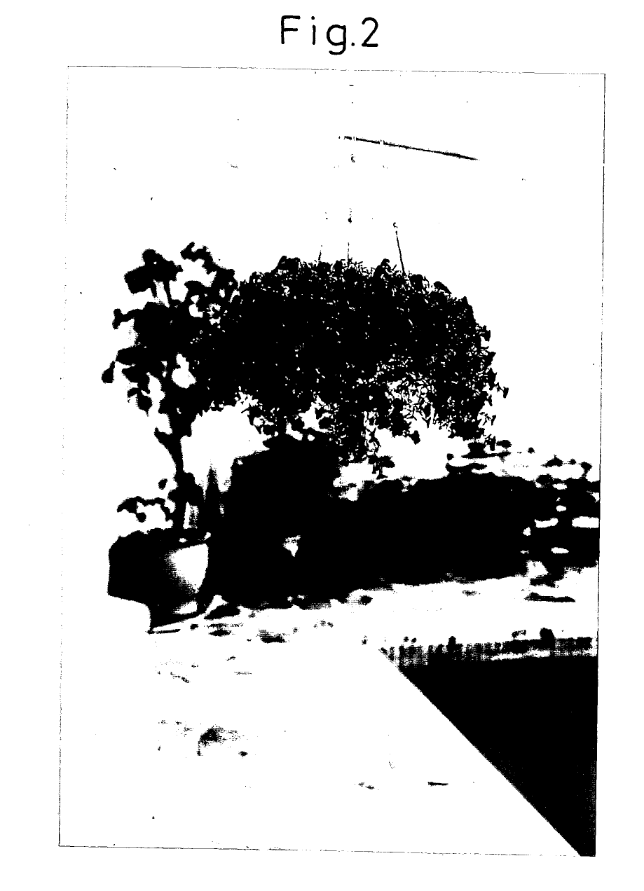

### DESCRIPTION OF THE NEW VARIETY

The botanical characteristics of the new and distinct variety of Petunia plant named ‘SUNBELRIKUPI’ are as follows when observing, during October at Youkaichi-shi, Shiga-ken, Japan, young plants that had been reproduced by cuttings in June.

Plant:

Growth habit.—Decumbent.

Plant height.—7.0 cm.

Spreading area of plant.—The stem extends to a length of 18 cm from the base.

Blooming period.—April to late October in the southern Kanto area, Japan. The plant shape does not change throughout this period.

Stem:

Thickness.—1.5 mm.

Pubescence.—Sparse.

Branching.—Extremely abundant with a superior branching propensity, especially secondary branches.

Length of internode.—1.8 cm.

Leaf:

Shape.—Lanceolate.

Length.—3.2 cm.

Width.—1.3 cm.

Color.—Moderate olive green (R.H.S. 147A, J.H.S. 3509).

Thickness.—0.5 mm.

Pubescence.—Sparse.

Leaf attaching angle to stem.—Horizontal.

Flower:

Facing direction.—Slanted upward.

Type.—Single.

Shape.—Funnel-shape, with five-fissured limb.

Shape of petal tip.—Round.

Waving of petal.—Weak.

Lobation of petal.—Shallow.

Diameter.—3.5 cm.

Color.—Petal: Vivid reddish purple (R.H.S. 74A, J.H.S. 9207). Bottom color of corolla throat: Brilliant greenish yellow (R.H.S. 8A, J.H.S. 2704). Outside color of corolla tube: Light greenish yellow (R.H.S. 5C, J.H.S. 2904).

Reproductive organs.—1 normal pistil and 5 normal stamens.

Fertility.—Fertile, but self-incompatible.

Peduncle.—0.9 mm in thickness, and 3.4 cm in length.

Physiological and ecological characteristics.—High resistance to heat, rain, drought and diseases such as powdery mildew. The resistance to heat and rain is very strong.

Blooming: A bloom commonly lasts approximately 10 days on the plant. Pinching is not necessary to ensure continuous blooming; however, it does tend to enhance bloom production.

This new variety of Petunia plant is most suitable for flower bedding and potting, particularly in hanging pots or planters, and is excellent for use as ground cover. Pinching of old blossoms will enhance the formation of new blossoms.

I claim:

1. A new and distinct variety of Petunia plant, substantially as herein illustrated and described, characterized particularly as to novelty by (A) having a decumbent growth habit, (B) great profusion of blooms with the entire plant remaining in bloom for a considerable period of time, (C) flowers are single and small, petals have a vivid reddish purple color, and (D) a high resistance to rain, heat, drought and disease.
